(19) United States
(12) Patent Application Publication
(10) Pub. No.: US 2026/0325065 A1
(43) Pub. Date: Oct. 22, 2026

(54) BIDIRECTIONAL SLEEP STUDY SYSTEM

(71) Applicant: Fascia Sleep Incorporated, Lynn, MA (US)

(72) Inventor: Guillermo Bernal, Lynn, MA (US)

(21) Appl. No.: 19/648,624

(22) Filed: Apr. 15, 2026

Related U.S. Application Data
(60) Provisional application No. 63/791,797, filed on Apr. 21, 2025.

(51) Int. Cl. A61B 5/00

(52) U.S. Cl. CPC A61B 5/4818; A61B 5/0022; A61B 5/0048; A61B 5/4812; A61B 5/6803; A61B 5/7203; A61B 5/7267

(57) ABSTRACT

A hub device for managing sleep of a patient includes a communication interface configured to communicate with a wearable sleep mask and a memory configured to store physiological data received from the wearable sleep mask. A controller determines, based on the physiological data and one or more locally generated or remotely received analysis results, at least one of a sleep state, a physiological condition, or a predicted disturbance of the patient. Based on the determination, the controller generates one or more control commands to cause modification of operation of the wearable sleep mask, modification of an environmental condition associated with the patient, or delivery of a stimulus to the patient. The hub device may operate in a closed-loop sleep management architecture in which physiological data is analyzed and used to modify monitoring and intervention during an ongoing sleep session.

### CROSS REFERENCE TO RELATED APPLICATIONS

This Application claims the benefit of U.S. Provisional Patent Application No. 63/791,797, filed Apr. 21, 2025, the contents of which are incorporated herein by reference in their entirety as if fully set forth.

### FIELD

Illustrative embodiments generally relate to sleep studies and, more particularly, various embodiments relate to controlling sleep study hardware and/or software.

### BACKGROUND

Sleep studies, known as polysomnography, are diagnostic tools used to monitor and evaluate physiological parameters during sleep. These studies are widely used to diagnose sleep disorders such as obstructive sleep apnea (OSA), insomnia, narcolepsy, and periodic limb movement disorder. Typical studies involve monitoring brain activity (EEG), eye movements (EOG), muscle activity (EMG), heart rhythm (ECG), airflow, respiratory effort, and oxygen saturation. As sleep disorders have been increasingly linked to conditions such as cardiovascular disease, diabetes, depression, and cognitive decline, the use of sleep studies has grown significantly.

Conventional sleep study systems are primarily passive, focusing on collecting physiological signals and generating post-hoc metrics such as sleep stages or apnea-hypopnea index (AHI). This approach places a burden on downstream analysis, as clinicians reconstruct sleep dynamics without contextual information captured during data acquisition. These systems also lack integration with other devices, patient-specific data, and adaptive capabilities, resulting in a one-size-fits-all methodology. Additionally, results are often delivered after significant delay and may require manual review, introducing cost, latency, and variability. Accordingly, existing systems emphasize data collection over real-time insight and intervention, limiting their effectiveness.

### SUMMARY OF VARIOUS EMBODIMENTS

In accordance with one embodiment, a hub device for managing sleep of a patient includes a communication interface configured to communicate with a wearable sleep mask and a memory configured to store physiological data received from the wearable sleep mask. A controller determines, based on the physiological data and one or more locally generated or remotely received analysis results, at least one of a sleep state, a physiological condition, or a predicted disturbance of the patient. Based on the determination, the controller generates one or more control commands to cause modification of operation of the wearable sleep mask, modification of an environmental condition associated with the patient, or delivery of a stimulus to the patient. The hub device may operate in a closed-loop sleep management architecture in which physiological data is analyzed and used to modify monitoring and intervention during an ongoing sleep session.

The hub device may further preprocess the physiological data prior to analysis to improve signal quality and reliability. Such preprocessing may include synchronizing physiological data from multiple sensor channels, buffering the physiological data, filtering noise, removing artifacts, and extracting features from the physiological data. The controller may also generate control commands to modify environmental conditions associated with the patient by controlling one or more environmental Internet-of-Things (IoT) devices, such as thermostats, lighting systems, speakers, humidifiers, or blinds, thereby dynamically adapting the patient's surroundings during sleep.

The hub device may further generate control commands to deliver one or more stimuli to the patient. The stimuli may include auditory, visual, olfactory, thermal, or vibrational inputs, enabling multi-modal interaction with the patient. In certain implementations, the hub device may generate control commands to cause coordinated delivery of a plurality of stimuli, including at least an auditory stimulus and an olfactory stimulus, to enhance combined physiological or cognitive effects. The controller may also determine an oscillatory phase of a physiological signal of the patient and generate control commands to deliver stimuli in a time-locked or phase-locked manner relative to the oscillatory phase, thereby aligning stimulation with physiological events to enhance effectiveness while minimizing disruption.

The hub device may continue receiving updated physiological data during a sleep session and iteratively update its analysis and generated control commands based on the updated data, thereby implementing a bidirectional closed-loop sleep monitoring and modification process. The hub device may also support automated setup and quality assurance functions, including automatically pairing with the wearable sleep mask, assessing signal quality of received physiological data, verifying proper fit of the wearable sleep mask, and generating feedback to improve data acquisition quality.

The communication interface may further communicate with additional devices beyond the wearable sleep mask, including modular sensor accessories, environmental control devices, or user interface devices, thereby expanding system functionality and available data sources. The hub device may also generate one or more representations of the physiological data using a machine learning model and determine the patient's sleep state, physiological condition, or predicted disturbance based on those representations, enabling advanced data-driven analysis and adaptive control of sleep-related interventions. The physiological condition may include, for example, motor parafunctions such as bruxism or position-dependent respiratory conditions such as positional obstructive sleep apnea.

In accordance with an additional embodiment, a method for managing sleep of a patient includes receiving physiological data from one or more sensors of a wearable sleep mask configured to be worn by the patient during sleep, receiving an analysis of the physiological data to determine at least one of a sleep state, a physiological condition, or a predicted disturbance of the patient, and receiving sleep information comprising one or more of analysis results, predictive information, model outputs, updated model parameters, control information, and intervention information. Based on the analysis of the physiological data and the sleep information, one or more control commands are forwarded to cause modification of operation of the wearable sleep mask, modification of an environmental condition, or delivery of a stimulus to the patient, thereby enabling adaptive management of sleep during an ongoing sleep session.

The method may further include preprocessing the physiological data prior to analysis to improve signal quality and reliability. Such preprocessing may include synchronizing physiological data from multiple sensor channels, buffering the physiological data, filtering noise, removing artifacts, and extracting features from the physiological data. The method may also include forwarding control commands to modify environmental conditions associated with the patient by controlling one or more environmental Internet-of-Things (IoT) devices, such as thermostats, lighting systems, speakers, humidifiers, or blinds, thereby dynamically adjusting the patient's environment.

The method may further include forwarding control commands to deliver one or more stimuli to the patient. The stimuli may include auditory, visual, olfactory, thermal, or vibrational inputs, enabling multi-modal interaction with the patient. The method may further include generating control commands to cause coordinated delivery of a plurality of stimuli, including at least an auditory stimulus and an olfactory stimulus. The method may also include determining an oscillatory phase of a physiological signal of the patient and forwarding control commands to deliver stimuli in a time-locked or phase-locked manner relative to the oscillatory phase, thereby coordinating stimulation with physiological activity.

The method may further include receiving updated physiological data during a sleep session and iteratively updating the analysis and the forwarded control commands based on the updated data, thereby implementing a bidirectional closed-loop sleep monitoring and modification process. The method may also include performing automated setup and quality assurance functions, including automatically pairing with the wearable sleep mask, assessing signal quality, verifying proper fit, and generating feedback to improve acquisition quality. The method may further include communicating with additional devices beyond the wearable sleep mask and generating one or more representations of the physiological data using a machine learning model to determine the sleep state, physiological condition, or predicted disturbance, including motor parafunctions or positional obstructive sleep apnea conditions.

In accordance with another embodiment, a system for managing sleep of a patient includes a wearable sleep mask configured to be worn by the patient during sleep, a hub device in communication with the wearable sleep mask, and a remote computing platform in communication with the hub device. The hub device is configured to receive physiological data from the wearable sleep mask, while the remote computing platform is configured to analyze the physiological data to determine at least one of a sleep state, a physiological condition, or a predicted disturbance of the patient and to generate sleep information. The hub device is further configured to receive the sleep information and forward one or more control commands based on the physiological data and the sleep information to cause modification of operation of the wearable sleep mask, modification of an environmental condition, or delivery of a stimulus to the patient.

The system may further include functionality for preprocessing physiological data, including synchronization, buffering, filtering, artifact removal, and feature extraction. The hub device may also control environmental Internet-of-Things (IoT) devices, such as thermostats, lighting systems, speakers, humidifiers, or blinds, to dynamically adjust environmental conditions associated with the patient.

The system may further be configured to deliver stimuli to the patient, including auditory, visual, olfactory, thermal, or vibrational stimuli. The hub device may coordinate delivery of multiple stimuli, including at least auditory and olfactory stimuli, and may determine an oscillatory phase of a physiological signal of the patient to coordinate delivery of stimuli in a time-locked or phase-locked manner relative to the oscillatory phase, thereby enhancing effectiveness of the intervention.

Illustrative embodiments are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments from the following “Description of Illustrative Embodiments,” discussed with reference to the drawings summarized immediately below.

FIG. 1 schematically shows an example bidirectional sleep study system in accordance with various embodiments.

FIG. 2 shows an example implementation of the bidirectional sleep study system of FIG. 1 in accordance with various embodiments.

FIG. 3 is a flow diagram of an example process/method for performing a bidirectional sleep study in accordance with an illustrative embodiment.

FIG. 4 is a block diagram schematically illustrating an example system architecture including a hub, a wearable device, and a cloud platform in accordance with various embodiments.

### DESCRIPTION OF ILLUSTRATIVE EMBODIMENTS

In illustrative embodiments, a bidirectional sleep study system monitors a patient's physiological state during sleep and, in response, generates control commands to modify the patient's sleep environment or deliver stimuli to the patient in real time. To that end, the system coordinates a wearable sleep mask, a local hub device, and a remote computing platform in a closed-loop architecture in which sensor data flows from the mask to a controlling hub, analysis occurs locally and/or remotely, and resulting control commands flow back to the mask or to other devices in the environment. Details of illustrative embodiments are discussed below.

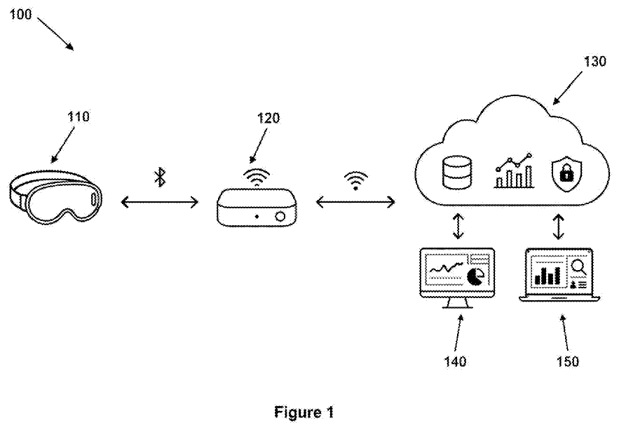

FIG. 1 schematically illustrates an example bidirectional sleep study system 100 in accordance with various embodiments. The system 100 is configured to monitor, analyze, and modify a patient's sleep in a closed-loop manner using coordinated hardware and software components.

The sleep study system 100 includes a sleep mask 110 configured to be worn by a patient during sleep and to collect physiological and related sensor data. In various embodiments, the sleep mask 110 includes one or more sensors forming an advanced multi-modal sensor array capable of collecting multiple physiological data streams. By way of example, and not limitation, the sensor array may include one or more of: electroencephalography (EEG) sensors (e.g., 5-channel EEG), electromyography (EMG) sensors (e.g., 2-channel EMG), electrooculography (EOG) sensors (e.g., 1-channel EOG), heart rate sensors, blood oxygen saturation (SpO2) sensors, skin temperature sensors, electrodermal activity sensors, acoustic sensors for snoring detection, respiration monitoring sensors, and inertial sensors (e.g., a 3-axis accelerometer and 3-axis gyroscope providing 6-axis motion sensing). These sensors collectively enable the sleep mask 110 to capture detailed information regarding the patient's neurophysiological, cardiovascular, respiratory, and behavioral states during sleep.

In addition to data acquisition, the sleep mask 110 in some embodiments may include onboard electronics configured for preliminary signal conditioning, such as amplification, filtering, and analog-to-digital conversion. The sleep mask 110 also may support pairing and communication protocols for transmitting collected data to other system components, as well as receiving control signals or instructions, as described further below.

The sleep study system 100 further includes a controlling hub 120 (hereinafter “hub 120”) in communication with the sleep mask 110. In some embodiments, the hub 120 communicates with the sleep mask 110 via a wireless interface, such as a low-energy wireless protocol (e.g., Bluetooth Low Energy compliant with IEEE 802.15 standards), although wired or alternative wireless communication techniques may be used. The hub 120 functions as a local Internet of Things (IoT) device that serves as an intermediary between the sleep mask 110 and remote computing resources.

The hub 120 may be configured to perform a variety of functions including device pairing, session initialization, signal quality monitoring, and user feedback. For example, the hub 120 may evaluate signal quality received from the sleep mask 110 and provide feedback to the user (e.g., via audio, visual, or haptic cues) to adjust the fit or placement of the mask to improve data acquisition. In various embodiments, the hub 120 may further perform preprocessing operations on the received data, such as synchronization of multiple data streams, artifact removal, feature extraction, and data buffering.

The hub 120 also may be configured to monitor environmental conditions in the patient's surroundings, including temperature, humidity, ambient light, and sound levels, using integrated or connected sensors. Based on these measurements, the hub 120 may interface with external or smart home devices (e.g., thermostats, lighting systems, speakers, or actuators) to modify environmental parameters. In this manner, the hub 120 participates in implementing a closed-loop system for sleep environment optimization.

In addition, the hub 120 may coordinate and deliver interventions directed to the patient. Such interventions may include, by way of example, auditory stimuli (e.g., tones, white noise, or guided audio), visual stimuli, vibrational feedback, thermal adjustments, or other forms of stimulation intended to influence sleep states or mitigate disturbances. These interventions may be triggered based on locally determined conditions, remotely provided instructions, or a combination thereof.

The hub 120 is further configured for communication with a cloud platform 130, which provides additional computational, analytical, and storage capabilities. The hub 120 may communicate with the cloud platform 130 using wireless communication protocols such as WiFi (e.g., IEEE 802.11), cellular communication, or other network technologies. Through this communication, the hub 120 may transmit raw or processed data to the cloud platform 130 and receive in return analysis results, predictive insights, updated models, or control instructions.

In some embodiments, the hub 120 may interface with additional devices beyond the sleep mask 110 via one or more application programming interfaces (APIs) or communication protocols. Such devices may include wearable devices (e.g., smartwatches, rings), bedding systems, environmental control devices, or other sensor platforms. The hub 120 may aggregate data from these devices and incorporate such data into its analysis and control processes, thereby enabling expanded monitoring and intervention capabilities.

In some embodiments, the system 100 may include or interface with an olfactory stimulation device configured to deliver scent-based stimuli to the patient. Such olfactory stimuli may include neutral, pleasant, or aversive odorants and may be delivered in a controlled manner during specific sleep stages. For example, olfactory stimuli may be delivered during non-REM sleep to influence memory consolidation or other cognitive processes, or may be delivered in response to detected physiological events (e.g., bruxism or movement patterns) as a form of behavioral conditioning. In some embodiments, olfactory stimulation may be coordinated with other modalities, such as auditory stimulation, to enhance overall effectiveness.

In some embodiments, the system 100 may detect motor parafunctions, such as bruxism, based on physiological data acquired from the wearable sleep mask 110. For example, bruxism events may be identified based on electromyography (EMG) signals indicative of rhythmic muscle activity, such as masseteric bursts associated with jaw clenching or grinding. Upon detection of such events, the system 100 may generate control commands to deliver an olfactory stimulus as part of a conditioning protocol.

The olfactory stimulus may comprise a mildly aversive, non-trigeminal odorant selected to avoid waking the patient while still being perceptible at a neural level. Such odorants may include compounds associated with unpleasant olfactory perception, and may be delivered in a temporally associated manner relative to the detected bruxism event. In this manner, the system may implement a conditioning approach that differs from conventional relaxation-based aromatherapy by associating a detected motor parafunction with an aversive stimulus to reduce or modify the behavior over time.

The system 100 may further coordinate delivery of multiple stimuli across different modalities in a temporally coordinated manner. For example, the hub 120 may generate control commands to cause simultaneous or coordinated delivery of an auditory stimulus and an olfactory stimulus to the patient. In certain embodiments, the auditory stimulus may be delivered in a time-locked or phase-locked manner relative to a detected physiological signal, while the olfactory stimulus is delivered in coordination with the auditory stimulus to enhance a combined physiological or cognitive effect. Such coordinated multi-modal stimulation may be used to influence sleep state dynamics, promote memory consolidation, or enhance responsiveness to stimuli. The timing, sequence, and intensity of each stimulus modality may be independently controlled and dynamically adjusted based on detected physiological conditions or predicted responsiveness of the patient.

In addition to the above-described stimulus modalities, some embodiments may include galvanic or electrical stimulation. For example, the sleep mask 110 or associated components may include one or more electrodes configured to deliver low-level electrical signals to a user. These signals may be applied transdermally (e.g., across the forehead or other contact regions) and may be used to influence neurological activity, promote sleep states, or enhance responsiveness to other stimuli. The electrical stimulation may be controlled by a controller (e.g., controller 116 described below) and may be coordinated with other stimulus modalities (e.g., audio, haptic, or visual stimuli) to achieve a desired physiological effect.

The cloud platform 130 may be configured to process incoming data from one or more hub devices 120, perform large-scale data aggregation, calculate sleep-related metrics, and execute advanced analytical or machine learning models. For example, the cloud platform 130 may perform sleep staging, detect sleep disturbances, predict future events, and generate recommendations or control signals. The cloud platform 130 also may maintain historical data and support longitudinal analysis across multiple sleep sessions.

In various embodiments, the cloud platform 130 and/or hub 120 may implement one or more machine learning architectures configured to generate latent representations of the patient's physiological state. For example, the system may include a foundation model configured to process heterogeneous, multi-modal physiological data streams (e.g., EEG, EOG, EMG, cardiac, respiratory, and environmental signals) and project such data into a shared latent representation space.

In certain implementations, the foundation model may generate embeddings of the physiological data, such as fixed-length vectors (e.g., on the order of hundreds of dimensions), that encode relationships across modalities and time. For example, the system may project multimodal physiological data into a shared latent space (e.g., a 128-dimensional latent space) to enable detection of subtle precursors to sleep disturbances and to support downstream tasks such as sleep staging, anomaly detection, and intervention selection. These latent representations, or embeddings, may capture cross-modal dependencies and temporal dynamics that are not readily observable from individual sensor signals alone.

The use of such embeddings may enable the system to perform unified analysis across diverse data types and to identify patterns indicative of emerging sleep disturbances prior to overt manifestation.

In some embodiments, the foundation model may include attention-based architectures, convolutional neural networks, recurrent neural networks, or combinations thereof, configured to perform cross-modal feature fusion. The model may generate patient-level embeddings, epoch-level embeddings, and/or event-level embeddings, which may be used for sleep staging, anomaly detection, disease risk prediction, and intervention selection. In this manner, the system may leverage learned representations rather than relying solely on handcrafted features or threshold-based logic.

In some embodiments, the system 100 may implement personalization mechanisms based on historical data associated with the patient. For example, embeddings generated across multiple nights may be compared to identify trends, deviations, or drift in physiological patterns. The system may update one or more models, thresholds, or intervention parameters based on observed responsiveness to prior interventions. In some embodiments, the system may generate patient-specific response profiles that characterize how the patient responds to different types of stimuli, thereby enabling adaptive adjustment of stimulation timing, intensity, and modality over time. Such adaptive learning may occur within a single sleep session or across multiple sessions.

In some embodiments, the system 100 may coordinate multiple types of stimuli across different modalities. For example, the system 100 may select among auditory, environmental, olfactory, visual, or haptic stimuli based on the detected physiological state of the patient. In certain embodiments, multiple stimuli may be delivered in combination, such as simultaneous auditory and environmental adjustments, or temporally coordinated sequences of stimuli. The selection, sequencing, and combination of stimuli may be determined based on detected sleep stage, predicted responsiveness, prior intervention outcomes, or user-specific preferences.

In various embodiments, the system 100 is configured to deliver stimuli at levels that do not substantially disrupt the user's sleep state. For example, the intensity, duration, and timing of stimuli (e.g., audio, haptic, visual, or electrical stimuli) may be calibrated to remain below a physiological arousal threshold, such as a K-complex arousal threshold. In this context, the K-complex threshold refers to a level of stimulation that may elicit a neural response without fully awakening the user.

Accordingly, the hub 120, the sleep mask 110, and/or the cloud platform 130 may dynamically adjust stimulus parameters based on sensed physiological data to maintain stimulation below such thresholds. This enables the system to influence sleep patterns, provide feedback, or deliver therapeutic stimuli while minimizing the likelihood of waking the user.

The system 100 may further include one or more user-facing interfaces, such as a clinician portal 140 and a researcher portal 150, which are configured to access data and analytical results provided by the cloud platform 130. Through these portals, clinicians and researchers may review sleep data, monitor patient status, analyze trends, configure study parameters, and provide or adjust intervention strategies. In some embodiments, these portals may also enable real-time or near-real-time interaction with the system, including sending commands or modifying protocols that are subsequently executed by the hub 120.

The bidirectional sleep study system 100 implements a tiered communication framework designed to provide reliability, security, and efficiency. For example, communication between the sleep mask 110 and the hub 120 may utilize low-power, short-range protocols optimized for wearable devices, while communication between the hub 120 and the cloud platform 130 may utilize higher-bandwidth, secure network protocols. Secure pairing, encryption, and authentication mechanisms may be employed at each communication layer to protect sensitive physiological data.

The system 100 supports bidirectional communication and closed-loop operation. Data collected by the sleep mask 110 is transmitted to the hub 120 and, in some cases, to the cloud platform 130 for analysis. Based on this data, the hub 120 and/or cloud platform 130 may generate control signals or intervention commands that are transmitted back to the hub 120 and/or sleep mask 110, or to other connected devices. This feedback loop allows the system to dynamically adjust monitoring parameters, environmental conditions, or patient-directed stimuli during an ongoing sleep session.

The architecture may further incorporate a distributed edge-cloud processing model, in which computational tasks are selectively performed at the hub 120 (edge) or in the cloud platform 130. For example, latency-sensitive operations, such as real-time detection of sleep state transitions or triggering of immediate interventions, may be performed locally at the hub 120. In contrast, computationally intensive tasks, such as model training or large-scale data analysis, may be performed in the cloud platform 130. The allocation of processing tasks between the hub 120 and cloud platform 130 may be dynamically determined based on factors such as latency requirements, available computational resources, privacy considerations, and network conditions.

Through this coordinated architecture, the bidirectional sleep study system 100 provides an integrated platform for continuous monitoring, analysis, and intervention, enabling more personalized, adaptive, and context-aware sleep assessment and optimization compared to conventional passive sleep study systems.

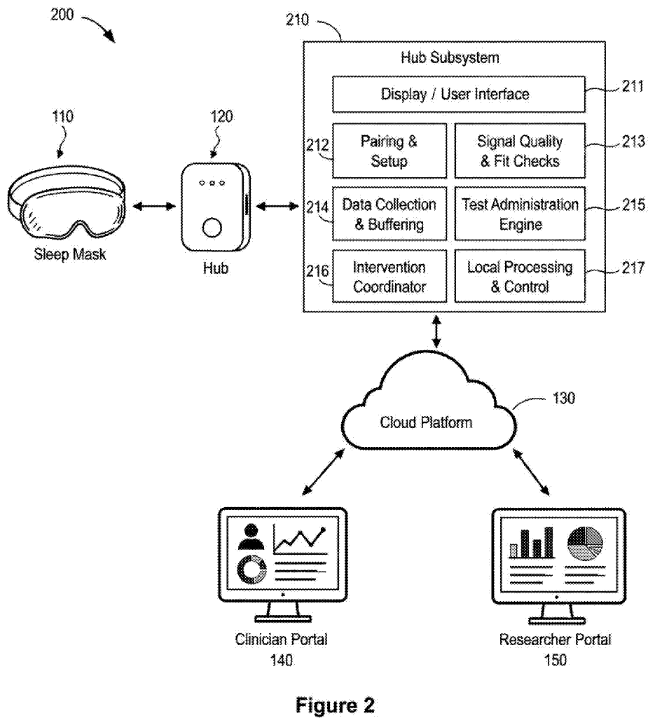

FIG. 2 illustrates an example implementation 200 of the bidirectional sleep study system 100 of FIG. 1 in accordance with various embodiments. In particular, FIG. 2 provides additional detail regarding internal functionality of the hub 120 and its role as a local control and orchestration device within the overall system architecture.

As shown, the hub 120 includes a subsystem 210, which may be implemented as a combination of hardware, firmware, and/or software components configured to perform the functions described herein. In various embodiments, the subsystem 210 includes one or more processors, memory, and associated circuitry configured to execute instructions corresponding to a plurality of functional components (also referred to herein as engines or modules). These components collectively enable the hub 120 to perform local data processing, system coordination, and closed-loop control operations.

The subsystem 210 may include a display and/or user interface component 211, which is configured to provide interaction between the system and the patient and/or other users. In some embodiments, the user interface component 211 may interface with a display device integrated into the hub 120 or the sleep mask 110, or with an external device such as a mobile application. The user interface component 211 may present information such as system status, signal quality indicators, instructions for device placement, or prompts related to sleep study setup. The user interface component 211 also may receive user inputs, such as responses to questionnaires, configuration selections, or acknowledgments.

The subsystem 210 further may include a pairing and setup component 212, which is configured to manage initialization and configuration of the system 100. For example, the pairing and setup component 212 may establish secure communication links between the hub 120 and the sleep mask 110, as well as any additional peripheral devices. The component 212 may also configure data acquisition parameters, select or customize sleep study protocols, and initialize system settings based on patient-specific information (e.g., questionnaire responses, demographic data, or prior sleep history).

To ensure reliable data acquisition, the subsystem 210 may include a signal quality and fit checks component 213. This component is configured to evaluate the quality of signals received from the sensors of the sleep mask 110. For example, the component 213 may analyze signal amplitude, noise levels, electrode impedance, or other metrics indicative of sensor contact and performance. Based on this analysis, the component 213 may generate feedback to the user (e.g., via audio, visual, or haptic cues) instructing the user to adjust the position or fit of the sleep mask 110 to improve signal quality.

The subsystem 210 also may include a data collection and buffering component 214, which is configured to receive physiological data from the sleep mask 110 and manage the storage and buffering of such data. In various embodiments, the component 214 may synchronize multiple data streams originating from different sensors, align them temporally, and temporarily store them for further processing or transmission. Buffering may be particularly useful to accommodate variations in communication bandwidth or to support intermittent connectivity with the cloud platform 130.

A test administration engine 215 may be included to manage execution of one or more sleep study protocols. In various embodiments, the test administration engine 215 may determine which data to collect, control timing and sequencing of measurements, and adjust parameters of the study dynamically based on observed conditions. For example, the engine 215 may modify sampling rates, enable or disable certain sensors, or initiate specific monitoring routines in response to detected sleep states or events.

The subsystem 210 further may include an intervention coordinator 216, which is configured to generate and coordinate interventions directed to the patient or the patient's environment. In various embodiments, the intervention coordinator 216 may receive inputs from local processing (e.g., component 217) and/or from the cloud platform 130 and determine appropriate intervention actions. For example, if the system detects entry into a particular sleep stage (e.g., slow-wave sleep), the intervention coordinator 216 may cause an auditory stimulus (e.g., a tone or noise pattern) to be delivered to the patient to enhance or prolong that sleep stage. Similarly, the intervention coordinator 216 may initiate environmental adjustments, such as modifying room temperature, lighting, or sound conditions via connected devices. In other scenarios, the intervention coordinator 216 may provide negative or corrective feedback (e.g., vibration or sound) to mitigate undesirable behaviors such as bruxism or restlessness.

A local processing and control component 217 may be provided to implement a processing pipeline for analyzing incoming data and generating control outputs. In various embodiments, the component 217 may perform signal preprocessing, feature extraction, and inference operations to determine the patient's current sleep state or detect anomalies. The component 217 may also integrate information received from the cloud platform 130, such as predictive models or analysis results, and combine this information with locally processed data to generate control signals. These control signals may then be used by the intervention coordinator 216 and other components to effect changes in system behavior, thereby enabling real-time or near-real-time closed-loop operation.

As illustrated, the hub 120 (and particularly subsystem 210) communicates with the cloud platform 130 and associated portals (e.g., clinician portal 140 and researcher portal 150) as described above with respect to FIG. 1. In this regard, the subsystem 210 may determine which data is processed locally versus transmitted to the cloud platform 130, thereby supporting a distributed edge-cloud processing architecture. Latency-sensitive operations, such as immediate intervention triggering, may be performed locally within subsystem 210, while more computationally intensive analyses or long-term model updates may be performed in the cloud platform 130.

It should be appreciated that the components illustrated within subsystem 210 are exemplary and that additional, fewer, or alternative components may be included depending on the specific implementation. For example, additional components may be included to support security, privacy, data compression, or integration with third-party devices and services. Similarly, the functionality of one or more components may be combined or distributed across multiple components without departing from the scope of various embodiments.

Through the coordinated operation of the components within subsystem 210, the hub 120 enables the bidirectional sleep study system 100 to move beyond passive data collection and to implement adaptive, context-aware monitoring and intervention during an ongoing sleep session.

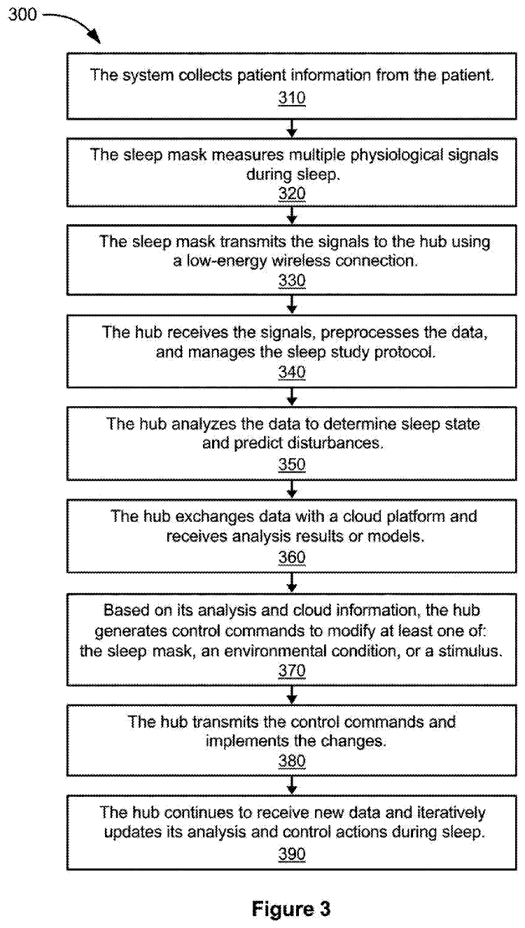

FIG. 3 is a flow diagram of an example process or method 300 for performing a bidirectional sleep study in accordance with an illustrative embodiment. The method 300 may be performed by the bidirectional sleep study system 100 described above with reference to FIGS. 1 and 2, including one or more of the sleep mask 110, hub 120, cloud platform 130, clinician portal 140, and researcher portal 150. In various embodiments, the method 300 implements a closed-loop process in which physiological and environmental data are acquired, analyzed, and used during an ongoing sleep session to modify monitoring, environmental conditions, and/or patient-directed stimuli.

At block 310, the system 100 collects patient information from the patient. In various embodiments, this patient information may be collected before the sleep session begins and may include demographic information, medical history, sleep history, medication information, questionnaire responses, target wake times, and/or user preferences. By way of example, the patient may provide questionnaire responses through a mobile application, portal, or other interface associated with the system. Such questionnaire data may include subjective sleepiness measures, sleep quality assessments, prior diagnoses, pre-sleep behavioral information, and other contextual information that may be used to configure or personalize the sleep study. In some embodiments, the collected patient information also may include prior-night or multi-night historical data associated with the patient, thereby allowing the system to initialize baseline models or individualized thresholds before acquisition of the current night's physiological data.

At block 320, the sleep mask 110 measures multiple physiological signals during sleep. As noted above, the sleep mask 110 may include a multimodal sensor array configured to acquire one or more of EEG, EMG, EOG, heart rate, SpO2, skin temperature, electrodermal activity, respiration-related signals, acoustic information, and inertial motion data. In certain embodiments, the sleep mask 110 may acquire these signals continuously or substantially continuously during the sleep session, including across multiple sleep stages such as wake, N1, N2, N3, and REM. In some implementations, the sleep mask 110 may additionally perform preliminary signal conditioning, such as amplification, digitization, or filtering, prior to transmission.

At block 330, the sleep mask 110 transmits the measured signals to the hub 120 using, for example, a low-energy wireless connection. In some embodiments, the connection may be a BLE or other short-range wireless protocol configured for low power consumption and secure streaming of biosensor data. Pairing and authentication procedures may be used to ensure secure association between the sleep mask 110 and the hub 120. In other embodiments, alternative wired or wireless communication protocols may be employed.

Upon receipt of the signals by the hub 120, the hub 120 preprocesses the data and manages the sleep study protocol at block 340. For example, the hub 120 may synchronize data streams received from different channels, buffer incoming data, filter noise, remove artifacts, and perform feature extraction. In some embodiments, preprocessing may include signal quality assessment, temporal alignment of heterogeneous sensor streams, motion artifact correction, and computation of preliminary time-domain and/or frequency-domain features. The hub 120 also may manage the sleep study protocol by selecting which sensors or data streams are active, adjusting sampling or analysis parameters, gating interventions, and/or configuring one or more tests based on the collected patient information and incoming physiological data. In some embodiments, the hub 120 may also evaluate mask fit or signal quality and provide feedback to the patient to improve acquisition quality.

At block 350, the hub 120 analyzes the data to determine the patient's current sleep state and predict disturbances or other events. For example, the hub 120 may classify or infer sleep stages, identify state transitions, estimate sleep depth, detect slow oscillations, determine whether the patient is in light sleep, deep sleep, or REM sleep, and detect or predict conditions such as arousals, respiratory events, snoring, movement-related events, bruxism, or other sleep disturbances. In some embodiments, the analysis may include generation of conventional or enhanced sleep metrics, such as total sleep time, sleep efficiency, sleep onset latency, wake after sleep onset, stage-specific durations, respiratory metrics, autonomic metrics, and other physiological or behavioral indicators. In further embodiments, the hub 120 may perform local real-time inference using one or more machine learning models, rule-based logic, state machines, or other decision systems to determine whether a particular intervention or adjustment is warranted. For example, the hub 120 may determine that stable N3 sleep has been reached, that a slow oscillation phase suitable for stimulation is approaching, or that an apnea-related event or arousal event requires suppression of stimulation.

In various embodiments, the analysis performed by the hub 120 and/or cloud platform 130 may include computation of a plurality of physiological, behavioral, and composite metrics. These metrics may include, by way of example, sleep architecture metrics (e.g., total sleep time, sleep efficiency, sleep stage durations), spectral metrics derived from EEG signals (e.g., slow-wave activity, spindle-related features), autonomic metrics (e.g., heart rate variability), respiratory metrics (e.g., apnea indices, oxygen desaturation), movement-related metrics, and environmental metrics. In addition, the system may compute composite or model-derived metrics, such as sleep quality scores, disease risk scores, or longitudinal trend indicators, based on combinations of these inputs.

The hub 120 and/or cloud platform 130 may determine the sleep state of the patient at a high temporal resolution. For example, in certain embodiments, sleep state classification may be performed at an epoch resolution of approximately five seconds. This higher temporal resolution, relative to conventional sleep staging approaches, enables more responsive detection of sleep state transitions and physiological events.

In some embodiments, the system is configured to perform analysis and generate control commands with sufficiently low latency to enable real-time or near-real-time intervention. For example, the system may achieve sub-100 millisecond latency between detection of a physiological condition and generation of a corresponding control command. Such low-latency processing enables precise timing of interventions, including phase-locked or time-sensitive stimuli. The hub 120 may further determine positional characteristics of the patient based on motion or inertial sensor data and identify position-dependent physiological conditions, including positional obstructive sleep apnea.

The hub 120 exchanges data with the cloud platform 130 and receives analysis results, model outputs, and/or updated models from the cloud platform 130 at block 360. In some embodiments, the hub 120 may transmit raw data, partially processed data, features, embeddings, event markers, or metric summaries to the cloud platform 130. The cloud platform 130 may perform additional or more computationally intensive analyses, such as longitudinal analysis across multiple nights, personalized model updating, disease-risk modeling, foundation-model inference, retrieval of protocol information, or generation of predictions and recommendations. The cloud platform 130 may then return information to the hub 120, such as updated thresholds, predicted responsiveness to stimulation, analysis results, intervention recommendations, or model parameters. In this regard, the method 300 may implement a distributed edge-cloud architecture in which latency-sensitive processing remains local to the hub 120 while more computationally intensive or longitudinal processing is performed remotely by the cloud platform 130.

In some embodiments, the system 100 may implement a mapping between detected physiological conditions and corresponding intervention responses. For example, detection of a particular sleep stage, oscillatory pattern, respiratory event, movement event, or environmental condition may be mapped to a corresponding action, such as delivering a stimulus, modifying an environmental parameter, or suppressing an ongoing intervention. This mapping may be predefined, dynamically learned, or adapted based on patient-specific data, thereby enabling context-aware and condition-specific responses.

At block 370, the hub 120 generates one or more control commands based on its local analysis and the information received from the cloud platform 130. These control commands may be used to modify the operation of one or more devices or otherwise influence the patient's sleep environment or physiological state. For example, the hub 120 may generate control commands to modify operation of the sleep mask 110, such as adjusting acquisition parameters or controlling a patient-facing interface. In other embodiments, the hub 120 may generate control commands to control one or more environmental devices, such as a thermostat, lights, blinds, speakers, humidifier, or other IoT-connected devices. In still other embodiments, the hub 120 may generate commands associated with a stimulus to be delivered to the patient, such as an auditory stimulus, masking sound, tone burst, pink-noise burst, white-noise signal, visual cue, olfactory cue, thermal adjustment, vibration, or another patient-directed intervention.

By way of example, if the system determines that the patient is in stable N3 sleep and a target slow oscillation phase is present, the hub 120 may generate a control command to deliver a brief auditory stimulus timed to the detected phase. In another example, if an apnea event, arousal, or environmental noise spike is detected, the hub 120 may generate a command to suppress ongoing auditory stimulation. In some embodiments, environmental noise may be detected and classified using audio signals captured by a microphone associated with the hub 120. For example, the system may analyze audio data using signal processing or machine learning techniques, such as spectral feature extraction (e.g., mel-spectrogram representations) and classification models, to identify ambient noise conditions. Based on this analysis, the system may determine a signal-to-noise ratio (SNR) or other measure of environmental interference and suppress or adjust stimulation when ambient noise exceeds a threshold or otherwise indicates that delivery of additional stimuli may compound disruption to the patient. For example, a convolutional neural network (CNN) or similar classifier may be used to distinguish environmental noise sources and assess their impact on stimulation effectiveness.

In still another example, if the patient has entered deep sleep, the hub 120 may generate a command to reduce ambient temperature via a connected thermostat or to initiate scent delivery in embodiments using olfactory stimulation hardware. Likewise, if a disturbance pattern is detected, the hub 120 may generate commands to initiate masking sound, adjust humidity, modify lighting, or provide a corrective or therapeutic intervention. The control commands generated at block 370 may therefore reflect immediate local analysis, cloud-assisted predictions, safety gating criteria, patient-specific protocols, and/or learned responsiveness profiles.

In some embodiments, one or more stimuli may be delivered in a time-locked or phase-locked manner relative to detected physiological signals. For example, the system may detect oscillatory brain activity (e.g., slow oscillations associated with deep sleep) and estimate an instantaneous phase of the oscillation in real time. Based on this phase estimate, the system may deliver a stimulus (e.g., an auditory signal) at a predetermined phase of the oscillation, such as an up-state of a slow oscillation. Such phase-locked stimulation may be configured to enhance or prolong desired sleep states, such as slow-wave sleep, while minimizing arousal risk.

In various embodiments, the system may implement gating or suppression logic to determine whether a stimulus should be delivered under current conditions. For example, the system may evaluate one or more gating conditions, such as detection of an arousal event, a respiratory disturbance, elevated noise levels, insufficient signal quality, or a sleep stage in which stimulation is undesirable. If one or more gating conditions are satisfied, the system may suppress or delay delivery of the stimulus. Conversely, when gating conditions indicate that stimulation is appropriate, the system may enable delivery of the stimulus. In this manner, the system may selectively provide interventions based on contextual and physiological conditions, thereby improving safety and efficacy.

In some embodiments, the oscillatory phase of the physiological signal may be determined using a real-time phase estimation algorithm. For example, the system may utilize an endpoint-corrected Hilbert transform (ecHT) to estimate the instantaneous phase of an oscillatory signal, such as a slow oscillation associated with deep sleep. The endpoint-corrected Hilbert transform enables accurate phase estimation in streaming or real-time conditions by compensating for boundary effects that arise in conventional Hilbert transform approaches.

By way of example, the system may apply the ecHT to a filtered representation of an electroencephalography (EEG) signal to determine the phase of slow oscillations during N3 sleep. Based on the estimated phase, the system may generate control commands to deliver a stimulus, such as an auditory stimulus, at a target phase (e.g., an up-state of the oscillation). This approach enables precise timing of stimulation to enhance desired physiological effects while minimizing disruption to the patient's sleep. Other phase estimation techniques may also be used, including analytic signal methods, adaptive filtering techniques, or model-based approaches, although the endpoint-corrected Hilbert transform may provide improved performance for real-time applications.

At block 380, the hub 120 transmits the control commands and implements the corresponding changes. In some embodiments, this may involve directly transmitting instructions to the sleep mask 110, to integrated output devices of the hub 120, or to one or more connected environmental or peripheral devices. For example, the hub 120 may cause a speaker to output an audio signal, cause a thermostat to lower or raise temperature, cause a light source to turn off or perform a dawn-simulation ramp, or cause a humidifier to adjust output. In some embodiments, implementation of the command may also include logging the intervention, recording contextual conditions at the time of the intervention, and monitoring whether the intervention was delivered successfully.

At block 390, the hub 120 continues to receive new data and iteratively updates its analysis and control actions during the sleep session. Thus, the method 300 is not limited to a one-time monitoring or intervention action, but instead may continuously or periodically reevaluate the patient's physiological state, environmental context, and prior intervention results. In this manner, the method 300 implements a bidirectional, closed-loop process in which the system both acquires information from the patient and acts on the patient and/or the patient's environment in response to that information. In some embodiments, the iterative updating performed at block 390 may include adaptive parameter modification across a single night, such as changing stimulation intensity, timing, frequency, density, or gating criteria based on observed effectiveness or arousal risk. In other embodiments, the iterative updating may also extend across multiple nights, such as by updating patient-specific models, response curves, baselines, or risk scores based on historical data collected over time.

In various embodiments, the method 300 also may incorporate safety and suppression logic. For example, stimulation may be paused or prevented if the system detects an arousal event, respiratory event, environmental disruption, insufficient signal quality, or another condition that renders stimulation undesirable. Likewise, the system may selectively enable particular interventions only during specific sleep stages, such as enabling phase-locked auditory stimulation during stable N3 sleep, enabling masking sound during lighter N1 or N2 sleep, or scheduling wake-promoting audio or light interventions near a target wake time. In some embodiments, the method 300 may additionally track and compute efficacy-related metrics associated with interventions, such as stimulation response scores, stimulation readiness scores, arousal rates, SWA enhancement, or other measures of intervention effect.

The operations and blocks of any method described herein, such as those of FIG. 3, are merely illustrative, and variations are contemplated to be within the scope of the present disclosure. For example, the order of one or more blocks may be changed, blocks may be combined or subdivided, and one or more blocks may be omitted or supplemented depending on the specific implementation. In addition, although various example components are described as performing specific functions, persons of ordinary skill in the art will appreciate that such functions may be implemented by other components, systems, or combinations thereof, including local processing at the hub 120, remote processing at the cloud platform 130, or distributed combinations of both, without departing from the scope of the present disclosure.

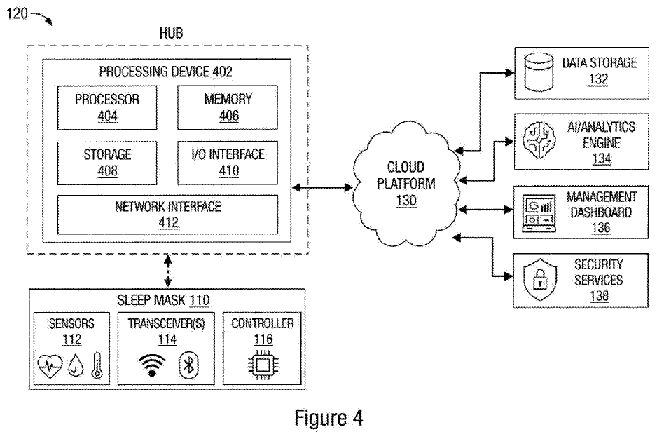

FIG. 4 schematically shows an example system architecture including a hub 120, a sleep mask 110, and a cloud platform 130 in accordance with various embodiments. The hub 120 is one example of a device in the system 100 that may be configured to perform one or more operations of process 300 illustrated in FIG. 3. The hub 120 includes a processing device 402 having various functional components, such as a processor 404, a memory 406, storage 408, an input/output interface 410, and a network interface 412.

The processing device 402 may be implemented as a programmable device, a dedicated hardwired state machine, or a combination thereof. For example, the processing device 402 may include one or more processors, Central Processing Units (CPUs), Digital Signal Processors (DSPs), or Field-Programmable Gate Arrays (FPGAs), among other things. In embodiments with multiple processing units, distributed, pipelined, or parallel processing may be used. The processing device 402 may execute programming instructions (e.g., software or firmware) stored in the memory 406 or storage 408, or may be at least partially implemented using dedicated hardware logic.

In various embodiments, the processor 404 (alone or in combination with other components of the processing device 402) may implement a controller configured to perform one or more control functions described herein. For example, the processor 404 may execute instructions stored in the memory 406 to determine, based on physiological data received from the sleep mask 110 and/or other sources, a sleep state, a physiological condition, or a predicted disturbance of a user, and to generate one or more control commands to modify operation of the sleep mask 110, adjust environmental conditions, or deliver one or more stimuli to the user.

The memory 406 and storage 408 may be implemented using one or more types of storage media, including volatile and/or non-volatile memory. For example, the memory 406 may include random access memory (RAM), while the storage 408 may include non-volatile storage such as flash memory, solid-state drives, or other persistent storage. These components may store programming instructions, operational data, and sensor data received from other components of the system.

The input/output interface 410 and network interface 412 enable the hub 120 to communicate with external devices and systems. For example, the network interface 412 may include wired or wireless communication interfaces (e.g., Ethernet, Wi-Fi, Bluetooth, cellular, or other communication protocols) that facilitate communication with the cloud platform 130 and other network-connected devices. The input/output interface 410 may include one or more ports, interfaces, or communication mechanisms for exchanging data with local devices.

The hub 120 is in communication with the sleep mask 110, which may be a wearable device configured to monitor physiological or environmental conditions of a user. The sleep mask 110 may include one or more sensors 112, one or more transceivers 114, and a controller 116. The sensors 112 may include, for example, biometric sensors (e.g., heart rate, motion, temperature, or other physiological sensors) or environmental sensors. The transceivers 114 may include wireless communication components configured to transmit data to and receive data from the hub 120. The controller 116 may process sensor data locally and manage operation of the sleep mask 110.

In addition to the sleep mask 110, some embodiments may include one or more modular sensor accessories configured to communicate with the hub 120. These accessories may be separate wearable or environmental devices that include sensors similar to or different from those of the sleep mask 110. For example, such accessories may include headbands, wrist-worn devices, chest straps, bedside sensors, or other devices configured to measure physiological or environmental parameters (e.g., heart rate, respiration, motion, temperature, or ambient conditions).

In some embodiments, the sleep mask 110 may include a removable or interchangeable front fascia. The front fascia may be configured to selectively permit or block light from reaching the user's eyes. For example, a first fascia may be configured as a blackout cover to substantially prevent ambient light from entering, while a second fascia may be at least partially transparent or translucent to allow the user to see through the mask.

The removable fascia may be attached using mechanical fasteners, magnetic couplings, or other attachment mechanisms, enabling the user to easily switch between configurations depending on use conditions or preferences. In this manner, the sleep mask 110 may be adapted for both sleep-focused and wakeful or augmented-reality-type applications.

These modular sensor accessories may communicate with the hub 120 via wired or wireless connections (e.g., Bluetooth, Wi-Fi, or other communication protocols) and may be selectively paired with the hub 120. In this manner, the system may be expanded or customized by adding or removing sensor modules depending on the application or user preferences. Data collected from these accessories may be processed locally by the hub 120 and/or transmitted to the cloud platform 130 for further analysis.

The hub 120 also communicates with the cloud platform 130 via the network interface 412. In some embodiments, the hub 120 may communicate with one or more additional devices, such as environmental control systems (e.g., lighting or climate systems), user interface devices, or other network-connected devices. The cloud platform 130 may provide various services, such as data storage 132, an analytics engine 134 (e.g., artificial intelligence or machine learning processing), a management dashboard 136, and security services 138.

The data storage 132 may store data received from the hub 120 and/or sleep mask 110. The analytics engine 134 may process and analyze the stored data to generate insights, predictions, or recommendations. The management dashboard 136 may provide a user interface for monitoring system status, configuring system parameters, and viewing data or analytics results. The security services 138 may provide authentication, authorization, encryption, or other security-related functions.

In operation, data collected by the sleep mask 110 may be transmitted to the hub 120, processed locally by the processing device 402, and selectively transmitted to the cloud platform 130 for storage, analysis, or further processing. Results or control instructions may be communicated back from the cloud platform 130 to the hub 120 and/or sleep mask 110. Although illustrated as separate components, one or more of the hub 120, sleep mask 110, and cloud platform 130 may be integrated or distributed across multiple devices or systems in various embodiments.

In various embodiments, the system may be configured to facilitate automatic setup and operation of the wearable sleep mask 110 and other associated devices. For example, the hub 120 may automatically pair with the sleep mask 110 and any modular sensor accessories upon detection. The hub 120 may further perform connection confirmation and signal-quality checks to ensure reliable communication between components. In response to determining that a signal quality or device placement is suboptimal, the hub 120 may generate one or more prompts or notifications to guide a user in adjusting the position or configuration of the sleep mask 110 or associated devices. Such prompts may be provided via a user interface, the management dashboard 136, or directly through the sleep mask 110 (e.g., via audio, visual, or haptic feedback). These features may be integrated into a core workflow to improve system reliability, user compliance, and data quality.

The architecture described above for system 100 may also include a hierarchical attention mechanism operating across varied temporal scales, enabling the identification of relationships between rapid physiological changes and longer-term behavioral patterns. Dynamic context windows adapt to the temporal characteristics of different phenomena, ensuring that the model appropriately considers relevant timeframes for different types of analysis and decision-making.

Contextual reasoning and causal inference capabilities can be implemented through probabilistic causal modeling that identifies potential sleep disturbance triggers. Feature extraction techniques identify subtle physiological precursors to sleep disturbances, while environmental correlation analysis links ambient conditions to sleep quality, enabling a comprehensive understanding of factors affecting sleep.

The technical infrastructure supporting this architecture can include a PyTorch 2.0implementation with custom compute unified device architecture (CUDA) kernels optimizing high-frequency signal processing. The training infrastructure employs four NVIDIA A100 GPUs, while inference is optimized to run efficiently on a single NVIDIA T4 GPU. The system achieves consistent sub-100 ms latency for 30-second epoch processing while maintaining 8GB memory utilization during inference, enabling real-time analysis and intervention.

In illustrative embodiments, the system provides comprehensive sleep staging capabilities through automated classification based on EEG, EMG, and EOG signals. This classification follows standardized sleep staging criteria while implementing enhanced detection of transitional states. The analysis includes identification of micro-arousals and brief awakenings that may not be perceived by the sleeper but can significantly impact sleep quality.

In various embodiments, the system facilitates contextually-relevant dialogue through session-based conversation initiation protocols that honor user privacy preferences. Pre-sleep behavioral assessment routines gather relevant daily activity data that can influence sleep quality, such as exercise, caffeine consumption, and stress levels.

Natural language processing techniques optimized for sleep-related dialogue enable the system to understand and respond to user queries and comments about sleep. Temporal association algorithms correlate conversation data with physiological sleep metrics, allowing the system to establish relationships between reported experiences and measured parameters.

Detection of sleep architecture abnormalities such as reduced slow-wave sleep or REM abnormalities provides insight into potential sleep disorders or disturbances. Sleep-disordered breathing assessment is accomplished through detection and classification of apnea and hypopnea events, including distinction between obstructive, central, and mixed apneas. Oxygen desaturation identification and quantification provides important information about the severity of breathing disturbances, while correlation of respiratory events with sleep fragmentation establishes connections between breathing problems and sleep disruption. Circadian rhythm analysis assesses sleep timing consistency and alignment with optimal circadian patterns, providing insight into potential circadian rhythm disorders or misalignment. The system identifies circadian misalignment contributing to sleep difficulties and provides recommendations for circadian rhythm optimization based on individual patterns and preferences.

The system may conduct daily activity impact analysis by correlating exercise timing and intensity with subsequent sleep quality. This analysis helps users understand how their physical activity affects their sleep and optimize their exercise schedules accordingly. Analysis of dietary patterns and their impact on sleep architecture provides insights into how nutrition affects sleep, particularly regarding substances like caffeine, alcohol, and specific nutrients. Stress and emotional state assessment in relation to sleep disruption helps users understand the psychological factors affecting their sleep quality. Environmental factor correlation identifies optimal environmental conditions for sleep quality based on individual responses to variables like temperature, humidity, light, and sound. The system assesses environmental disruptors impacting sleep continuity and provides personalized recommendations for environmental optimization based on detected patterns and preferences.

The system implements dynamic intervention planning through strategy selection based on identified causal mechanisms. This approach ensures that interventions address the specific factors disrupting sleep rather than applying generic solutions. Personalized intervention development incorporating historical effectiveness ensures that strategies evolve based on their observed impact for each individual. Natural language generation for targeted audio guidance creates customized verbal interventions for issues such as difficulty falling asleep or nighttime awakenings. Environmental control capabilities include temperature, humidity, sound, and lighting adjustments based on physiological indicators. These adjustments can be implemented automatically or suggested to the user, depending on system configuration and user preferences. A closed-loop validation system measures intervention effectiveness, allowing the system to refine its strategies based on observed outcomes. Personalized environmental profile development ensures that adjustments align with individual preferences and needs. The system also provides behavioral guidance through personalized recommendations for pre-sleep routine optimization. These recommendations are based on observed correlations between evening activities and subsequent sleep quality. Cognitive Behavioral Therapy for Insomnia (CBT-I) support provides elements of this evidence-based therapy in an accessible format, helping users address insomnia symptoms without formal therapy sessions. Biofeedback-guided relaxation techniques utilize physiological measurements to guide users in effective relaxation practices that promote sleep onset and quality.

The bidirectional AI communication system creates a dynamic interaction framework between the user and system that extends beyond passive monitoring to active engagement and intervention, representing a significant advancement in sleep technology. This capability enables more effective user education, engagement, and intervention implementation.

Various embodiments may be implemented at least in part in any conventional computer programming language. For example, some embodiments may be implemented in a procedural programming language (e.g., “C”), or in an object-oriented programming language (e.g., “C++”). Other embodiments may be implemented as a pre-configured, stand-alone hardware element and/or as preprogrammed hardware elements (e.g., application specific integrated circuits, FPGAs, and digital signal processors), or other related components.

In an alternative embodiment, the disclosed apparatus, and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical, or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments may be implemented as a combination of both software (e.g., a computer program product) and hardware. Still other embodiments are implemented as entirely hardware, or entirely software.

The embodiments described above are intended to be merely exemplary; numerous variations and modifications will be apparent to those skilled in the art. Such variations and modifications are intended to be within the scope of various embodiments.

What is claimed is:

1. A hub device for managing sleep of a patient, the hub device comprising:
a communication interface configured to communicate with a wearable sleep mask;
a memory configured to store physiological data received from the wearable sleep mask; and
a controller configured to determine, based on the physiological data, and further based on one or more locally or remotely generated received results of an analysis of the physiological data, at least one of a sleep state, a physiological condition, or a predicted disturbance of the patient,
the controller further configured to generate, based on the determination, one or more control commands to cause at least one of modification of operation of the wearable sleep mask, modification of an environmental condition associated with the patient, or delivery of a stimulus to the patient.

2. The hub device of claim 1, wherein the controller is further configured to perform preprocessing of the physiological data, the preprocessing comprising one or more of: synchronizing physiological data from multiple sensor channels, buffering the physiological data, filtering noise from the physiological data, removing artifacts from the physiological data, or extracting features from the physiological data.

3. The hub device of claim 1, wherein the one or more processors are further configured to generate the one or more control commands to cause modification of the environmental condition by controlling one or more environmental Internet-of-Things (IoT) devices associated with the patient.

4. The hub device of claim 3, wherein the one or more IoT devices include one or more of: a thermostat, a lighting device, a speaker, a humidifier, or a blind.

5. The hub device of claim 1, wherein the one or more processors are further configured to generate the one or more control commands to cause delivery of the stimulus to the patient, the stimulus comprising at least one of an auditory stimulus, a visual stimulus, an olfactory stimulus, a thermal stimulus, or a vibrational stimulus.

6. The hub device of claim 5, wherein the one or more processors are further configured to generate the one or more control commands to cause coordinated delivery of a plurality of stimuli comprising at least an auditory stimulus and an olfactory stimulus to the patient.

7. The hub device of claim 5, wherein the one or more processors are further configured to determine an oscillatory phase of a physiological signal of the patient and generate the one or more control commands to cause delivery of the stimulus in a time-locked or phase-locked manner relative to the oscillatory phase.

8. The hub device of claim 1, wherein the one or more processors are further configured to continue receiving updated physiological data during a sleep session and iteratively update the analysis of the physiological data and the one or more control commands based on the updated physiological data to implement a bidirectional closed-loop sleep monitoring and modification process.

9. The hub device of claim 1, wherein the controller is further configured to perform one or more of the following: automatically pair with the wearable sleep mask, assess signal quality of physiological data received from the wearable sleep mask, verify fit of the wearable sleep mask based on the physiological data, or generate feedback to a patient to improve acquisition quality of the physiological data.

10. The hub device of claim 1, wherein the communication interface is further configured to communicate with one or more additional devices other than the wearable sleep mask, the one or more additional devices including at least one of a modular sensor accessory, an environmental control device, or a user interface device.

11. The hub device of claim 1, wherein the controller is further configured to generate one or more representations of the physiological data using a machine learning model and to determine the at least one of the sleep state, physiological condition, or predicted disturbance based on the one or more representations.

12. The hub device of claim 1, wherein the physiological condition comprises at least one of a motor parafunction or a positional obstructive sleep apnea condition.

13. A method for managing sleep of a patient, the method comprising:
receiving physiological data from one or more sensors of a wearable sleep mask configured to be worn by the patient during sleep;
receiving an analysis of the physiological data to determine at least one of a sleep state, a physiological condition, or a predicted disturbance of the patient;
receiving, sleep information comprising one or more of analysis results, predictive information, model outputs, updated model parameters, control information, and intervention information; and
forwarding one or more control commands to cause one or more of modification of operation of the wearable sleep mask, modification of an environmental condition, and delivery of a stimulus to the patient, the one or more control commands based on the analysis of the physiological data and the sleep information.

14. The method of claim 13, further comprising preprocessing the physiological data, the preprocessing comprising one or more of: synchronizing physiological data from multiple sensor channels, buffering the physiological data, filtering noise from the physiological data, removing artifacts from the physiological data, or extracting features from the physiological data.

15. The method of claim 13, wherein forwarding the one or more control commands comprises causing modification of the environmental condition by controlling one or more environmental Internet-of-Things (IoT) devices associated with the patient.

16. The method of claim 15, wherein the one or more IoT devices include one or more of: a thermostat, a lighting device, a speaker, a humidifier, or a blind.

17. The method of claim 13, wherein forwarding the one or more control commands comprises causing delivery of the stimulus to the patient, the stimulus comprising at least one of an auditory stimulus, a visual stimulus, an olfactory stimulus, a thermal stimulus, or a vibrational stimulus.

18. The method of claim 17, further comprising generating the one or more control commands to cause coordinated delivery of a plurality of stimuli comprising at least an auditory stimulus and an olfactory stimulus to the patient.

19. The method of claim 17, further comprising determining an oscillatory phase of a physiological signal of the patient and forwarding the one or more control commands to cause delivery of the stimulus in a time-locked or phase-locked manner relative to the oscillatory phase.

20. The method of claim 13, further comprising receiving updated physiological data during a sleep session and iteratively updating the analysis of the physiological data and the one or more control commands based on the updated physiological data to implement a bidirectional closed-loop sleep monitoring and modification process.

21. The method of claim 13, further comprising performing one or more of the following: automatically pairing with the wearable sleep mask, assessing signal quality of physiological data received from the wearable sleep mask, verifying fit of the wearable sleep mask based on the physiological data, or generating feedback to a patient to improve acquisition quality of the physiological data.

22. The method of claim 13, further comprising communicating with one or more additional devices other than the wearable sleep mask, the one or more additional devices including at least one of a modular sensor accessory, an environmental control device, or a user interface device.

23. The method of claim 13, further comprising generating one or more representations of the physiological data using a machine learning model and determining the at least one of the sleep state, physiological condition, or predicted disturbance based on the one or more representations.

24. The method of claim 13, wherein the physiological condition comprises at least one of a motor parafunction or a positional obstructive sleep apnea condition.

25. A system for managing sleep of a patient, the system comprising:
a wearable sleep mask configured to be worn by the patient during sleep, the wearable sleep mask comprising one or more sensors configured to generate physiological data;
a hub device in communication with the wearable sleep mask; and
a remote computing platform in communication with the hub device;
wherein:
the hub device is configured to receive the physiological data from the wearable sleep mask;
the remote computing platform is configured to analyze the physiological data to determine at least one of a sleep state, a physiological condition, or a predicted disturbance of the patient and to generate sleep information comprising one or more of analysis results, predictive information, model outputs, updated model parameters, control information, and intervention information; and
the hub device is further configured to receive the sleep information from the remote computing platform and to forward one or more control commands based on the analysis of the physiological data and the sleep information to cause one or more of modification of operation of the wearable sleep mask, modification of an environmental condition, and delivery of a stimulus to the patient.

26. The system of claim 25, wherein the hub device is further configured to preprocess the physiological data, the preprocessing comprising one or more of: synchronizing physiological data from multiple sensor channels, buffering the physiological data, filtering noise from the physiological data, removing artifacts from the physiological data, or extracting features from the physiological data.

27. The system of claim 25, wherein the hub device is further configured to forward the one or more control commands to cause modification of the environmental condition by controlling one or more environmental Internet-of-Things (IoT) devices associated with the patient.

28. The system of claim 27, wherein the one or more IoT devices include one or more of: a thermostat, a lighting device, a speaker, a humidifier, or a blind.

29. The system of claim 25, wherein the hub device is further configured to forward the one or more control commands to cause delivery of the stimulus to the patient, the stimulus comprising at least one of an auditory stimulus, a visual stimulus, an olfactory stimulus, a thermal stimulus, or a vibrational stimulus.

30. The system of claim 29, wherein the hub device is further configured to determine an oscillatory phase of a physiological signal of the patient and to forward the one or more control commands to cause delivery of the stimulus in a time-locked or phase-locked manner relative to the oscillatory phase.
